(19) United States
(12) Patent Application Publication
(10) Pub. No.: US 2003/0170207 A1
(43) Pub. Date: Sep. 11, 2003

(54) Stable aqua formulation of interferon, the preparation method and the uses thereof

(76) Inventor: Lei Zhang, Tianjin (CN)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/148,865

(22) PCT Filed: Dec. 5, 2000
(86) PCT No.: PCT/CN00/00531

(30) Foreign Application Priority Data
Dec. 6, 1999 (CN) 99125582.2

(51) Int. Cl. A61K 38/21; A61K 31/721

(52) U.S. Cl. 424/85.7; 514/59

(57) ABSTRACT

The present invention relates to a stable interferon alpha aqueous solution formulation that may be preservative or human blood extracted ingredient free, which comprises of the following components:

a. interferon alpha;

b. a buffer system to maintain pH 4.5-9.0;

c. a stabilizing agent;

d. a nonionic surfactant;

e. an osmotic pressure regulator;

the preparation method and the uses in the treatment of viral, tumorous and immune diseases thereof.

### FIELD OF THE INVENTION

The present invention relates to a stable interferon alpha aqueous solution formulation that may be preservative or human blood extracted ingredient free, the preparation method and uses thereof.

### DESCRIPTION OF THE BACKGROUND ART

Interferon (IFN) has broad-spectrum antiviral, antitumor and immunomodulatory activities. It has been widely used clinically for the treatment of a variety of viral and tumorous diseases, such as acute and chronic viral hepatitis B, hepatitis C, condylomata acuminata, chronic granulocytic leukemia and lymphoma, etc. In recent years, due to the high manufacturing cost and the inconvenience in clinical use of lyophilized products, as well as the possibility of drug contamination induced by the use of disqualified syringes or injection water, the wider use of interferon has been limited. Therefore, the research of stable aqueous interferon injection formulation has been paid a lot of attention to.

Most of the stable formulations of interferon in market contain human blood extracted ingredients, such as human serum albumin, as the stabilizer. Unfortunately, use of human serum albumin complicates the production process, and especially brings the hazard of blood product contamination and virus infection.

Chinese Patent Application Publications CN 1160355A and CN 1141808A disclosed two solution formulations of interferon without human serum albumin. However, the preservative normally has more or less toxicity and side effects. The formulations containing preservative may bring certain kinds of stimulations to the injection site during injection. Further, most preservatives affect the bioactivity of interferon. Thus, a stable interferon aqueous solution formulation which is preservative or human blood extracted ingredient free is in need.

### SUMMARY OF THE INVENTION

In one aspect, the present invention provides a stable interferon aqueous solution formulation that may be preservative or human blood extracted ingredient free, which comprises the following components:

1. an interferon alpha;

2. a buffer system to maintain pH 4.5-9.0;

3. an interferon stabilizer;

4. a nonionic surfactant;

5. an osmotic pressure regulator;

6. injection water.

In another aspect, the invention provides a preparation method for the above-described stable interferon aqueous solution formulation that may be preservative or human blood extracted ingredient free, said method comprising the step of admixing interferon alpha, the buffer system to maintain pH 4.5-9.0, the interferon stabilizer, the nonionic surfactant, the isotonicity agent, and appropriate amount of injection water to form an aqueous formulation.

In still another aspect, the invention provides a use of interferon alpha, a buffer system to maintain pH 4.5-9.0, an interferon stabilizer, a nonionic surfactant and an isotonicity agent, in the preparation of the above-described stable interferon aqueous formulation that may be preservative or human blood extracted ingredient free.

In still another aspect, the invention provides a use of the above-described stable interferon aqueous solution formulation that may be preservative or human blood extracted ingredient free, in the treatment of viral, tumorous and immune diseases.

In still another aspect, the invention provides a therapeutic method of viral, tumorous and immune diseases with the above-described stable interferon aqueous solution formulation that may be preservative or human blood extracted ingredient free, said method comprising the step of administrating an effective amount of said formulation to the patients in need.

In still another aspect, the invention provides a medicament with pre-filled syringe, said pre-filled syringe is filled with an effective amount of said stable interferon aqueous solution formulation of the present invention.

Other aspects and advantages of the invention will be apparent to those skilled in the art from the following detailed description of the invention. For example, in the various detailed systems of the stable interferon aqueous solution formulation according to the present invention, by means of addition of various different excipients directed to other formulations, which are generally recognized in the art, various kinds of stable formulations of interferon that have the advantages of the present invention, such as tablets, lotions, ointments, suppositories, oral sprays, liposome, eyedrops, PEG-interferon, etc, can be prepared. In addition, those skilled in the art can prepare other stable bioactive protein systems based on the various specific systems of the present invention.

### DETAILED DESCRIPTION OF THE INVENTION

A stable interferon aqueous solution formulation that may be preservative or human blood extracted ingredient free in accordance with the present invention, comprises the following components:

1. interferon alpha;

2. a buffer system to maintain pH 4.5-9.0;

3. an interferon stabilizer;

4. a nonionic surfactant;

5. an osmotic pressure regulator;

6. injection water.

Said formulation, after storage at 2-10° C. for 24 months, can still maintain high biological and physical stability of interferon alpha.

Preferably, a stable interferon aqueous solution formulation that may be preservative or human blood extracted ingredient free according to the present invention comprises the following components:

1. 100,000-100,000,000 IU/ml of interferon alpha;

2. a buffer solution of citric acid and disodium hydrogen phosphate to maintain pH 4.5-9.0;

3. 5-60mg/ml of hydroxyethyl starch;

4. 0.02-0.2 ml/100 ml of polyoxyethylene sorbitan monooleate;

5. 1-10 mg/ml of sodium chloride and 10 mg/ml of mannitol;

6. appropriate amount of injection water.

Said formulation, after storage at 2-10° C. for 24 months, can still maintain high biological and physical stability of interferon alpha.

The term “May be preservative or human blood extracted ingredient free” according to the present invention has the meaning that during the preparation process of said aqueous formulation, preservative and human blood extracted ingredient are applied or not applied. “Preservative” and “human blood extracted ingredient” according to the present invention have their generally recognized meanings in the art, such as phenol and human serum albumin, respectively.

The term “Interferon alpha” according to the present invention is any type of natural or recombinant interferon alpha and derivative thereof which has antiviral, anticancer and immunomodulatory activities, which is industrially manufactured and clinically applied, including but not limited to interferon alpha-2b, PEG-interferon, etc.

The term “Buffer system to maintain pH 4.5-9.0” according to the present invention is any kind of buffer system that is capable of maintaining the pH of aqueous formulation at 4.5-9.0, including phosphoric acid, citric acid, disodium hydrogen phosphate (Na₂HPO₄), sodium dihydrogen phosphate (NaH₂PO₄), etc and the mixtures thereof. The buffer system of citric acid and Na₂HPO₄ is preferred, because the pH range of these two components after combination is identical to the pH value of human internal environment. Further, the components of this system also have metal ion chelating and self-oxidation prevention effects.

The term “Stabilizing agent” according to the present invention is any generally recognized plasma substitute which is human blood extracted ingredient free in the art, including but not limited to hydroxyethyl starch 40, dextran, etc, preferably hydroxyethyl starch 40. With the existence of the stabilizing agent, the dimensional structure of interferon molecule and the activity thereof can be stabilized, and the oxidation of interferon can be prevented, without the necessity of inflation of nitrogen during the procedure of preparing drug solution. The content of stabilizing agent in the formulation, when the formulation of present invention contains 100,000-100,000,000 IU/ml of interferon, is 5-60 mg/ml, preferably 10-20 mg/ml.

The term “Nonionic surfactants” according to the present invention is any generally recognized nonionic surfactant in the art, including but no limited to polyoxyethylene sorbitan monooleate (Tween 80), Tween 20, Span 80, etc. Such surfactants act as suspending agent for interferon and prevent protein aggregation. The content of surfactants in the formulation is 0.02-0.2 ml/100 ml, preferably 0.1 ml/100 ml.

The term “Osmotic pressure regulator” according to the present invention is any generally recognized osmotic pressure regulator in the art, including not limited to sodium chloride (NaCl), mannitol, glycerin, etc. Preferably, NaCl and mannitol. Preferably, 4 mg/ml of NaCl and 10 mg/ml of mannitol.

The term “Maintain high biological and physical stability of interferon alpha” according to the present invention means that said formulation of present invention can maintain at least 70%, preferably at least 85%, more preferably at least 95%, and most preferably 100%, of its bioactivity, after storage at 2-10° C. for at least 24 months, preferably at least 36 months (cf. the Examples, and European Pharmacopoeia 1997, third edition, Determination Method of Interferon Polency, p1031-1035, for the determining method of the antiviral activity). And in the same time, the appearance maintains colorless and transparent without protein aggregation and bacteria contamination.

The aqueous interferon alpha solution formulation of present invention has surprising excellent stability. Particularly, said formulation, after first storage at room temperature for at least 8 weeks and then at 2-10° C. for at least 24 months, still maintains high biological and physical stability of interferon alpha. Moreover, comparing with other currently using aqueous interferon alpha solution formulations, in view of increasing the tolerance against freezing-thawing, temperature and shaking, as well as decreasing the stimulation to the injection site, etc, we have taken into account in the formulation design and such are shown in the results of experiments.

The method for preparing the aforementioned stable interferon aqueous solution formulation that may be preservative or human blood extracted ingredient free of present invention, comprises the following steps:

dissolving appropriate amount of buffer at pH 4.5-9.0, stabilizing agent, nonionic surfactants and isotonicity agent in an appropriate amount of injection water, which is sterilized and pyrogen free; adding a predetermined amount of interferon alpha substance and diluting such to an appropriate concentration; sterile membrane filtering; filling, etc.

The aqueous interferon solution formulation of the present invention can also be a pre-filled form in a sterile syringe with a precise amount. Such formulation is more convenient for clinical administration.

Furthermore, within the ability of those skilled in the art, without any inventive work, other stable formulations of interferon, such as tablets, lotions, ointments, suppositories, oral sprays, liposome, eyedrops and PEG-interferon and so on, can be achieved, by means of adding different generally recognized excipients in the art; further, other stable bioactive protein systems can also be achieved.

The following Examples are provided to illustrate the invention and do not limit it in any way.

### EXAMPLES

### Example 1 (pH: 7.0)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.4 | g, |
| dextran | 2 | g, |
| Tween 80 | 0.1 | ml, |
| mannitol | 1 | g, |
| injection water | to 100 | ml. |

All the components except interferon were measured and solved with bacteria and pyrogen free injection water. Interferon alpha-2b substance was added and diluted to an appropriate concentration. The solution was sterile filtered with 0.22 μm filtering membrane, stored at 2-8° C. Samples were collected for sterility and pyrogen assay. If passed, the solution was filled and finished into sealed container in the 100-Class Clean Region. The products were stored at 2-8° C. in dark.

### Example 2 (pH: 7.1)

Composition:

| | |
|---|---|
| recombinant interferon alpha-2b | 1 × 10⁸IU, |

| | | |
|---|---|---|
| citric acid | 0.02 | g, |
| Na₂HPO₄ | 0.25 | g, |
| NaCl | 0.4 | g, |
| hydroxyethyl starch 40 | 2 | g, |
| Tween 20 | 0.1 | ml, |
| mannitol | 1 | g, |
| injection water | to 100 | ml. |

Preparation method was same as described in Example 1.

### Example 3 (pH: 5.0)

Composition:

| | |
|---|---|
| recombinant interferon alpha-2b | 5 × 10⁸IU, |

| | | |
|---|---|---|
| citric acid | 0.102 | g, |
| Na₂HPO₄ | 1.46 | g, |
| NaCi | 0.4 | g, |
| hydroxyethyl starch 40 | 5 | g, |
| Tween 20 | 0.1 | ml, |
| phenol | 0.4 | g, |
| glycerin | 6.3 | g, |
| injection water | to 100 | ml. |

Preparation method was same as described in Example 1.

### Example 4 (pH: 7.1)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.4 | g, |
| hydroxyethyl starch 40 | 1 | g, |
| Span 20 | 0.1 | ml, |
| injection water | to 100 | ml. |

Preparation method was same as described in Example 1.

### Example 5 (pH: 8.8)

Composition:

| recombinant interferon alpha-2b | 8 × 10⁸IU, | |
|---|---|---|
| glycine (0.2 M) | 25 | ml (equivalent to 0.375 g), |
| NaOH (0.2 M) | 2 | ml, |
| dextran | 2 | g, |
| Tween 80 | 0.1 | ml, |
| injection water | to 100 | ml. |

Preparation method was same as described in Example 1.

### Example 6: (pH: 6.6)

Composition:

| | |
|---|---|
| recombinant interferon alpha-2b | 5 × 10⁶IU, |

| | | |
|---|---|---|
| NaH₂PO₄ | 0.435 | g, |
| Na₂HPO₄ | 0.675 | g, |
| NaCl | 0.425 | g, |
| hydroxyethyl starch 40 | 3 | g, |
| Tween 20 | 0.1 | ml, |
| injection water | to 100 | ml. |

Preparation method was same as described in Example 1.

### Example 7: (pH: 7.1)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.4 | g, |
| PVP | 5 | g, |
| Tween 80 | 0.1 | ml, |
| injection water | to 100 | ml. |

Preparation method was same as described in Example 1.

### Example 8: (pH: 7.1)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.4 | g, |
| hydroxyethyl starch 40 | 1 | g, |
| dextran | 1 | g |
| Tween 80 | 0.1 | ml, |
| injection water | to 100 | ml. |

Preparation method was same as described in Example 1.

### Example 9: (pH: 7.0)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.4 | g, |
| PVP | 1 | g |
| dextran | 1 | g, |
| Tween 80 | 0.1 | ml, |
| injection water | to 100 | ml. |

Preparation method was same as described in Example 1.

### Example 10: (pH: 7.2)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.4 | g, |
| hydroxyethyl starch 40 | 1 | g, |
| PVP | 1 | g |
| Tween 80 | 0.1 | ml, |
| injection water | to 100 | ml. |

Preparation method was same as described in Example 1.

### Example 11: (pH: 7.0)

Composition:

| | |
|---|---|
| recombinant interferon alpha-2b | 3 × 10⁸IU, |

| | | |
|---|---|---|
| citric acid | 0.2 | g, |
| Na₂HPO₄ | 0.25 | g, |
| NaCl | 0.4 | g, |
| dextran | 2 | g |
| Tween 80 | 0.1 | ml, |
| injection water | to 100 | ml. |

Preparation method was same as described in Example 1.

### Example 12: (pH: 7.0)

Composition:

| | |
|---|---|
| recombinant interferon alpha-2b | 3 × 10⁸IU, |

| | | |
|---|---|---|
| citric acid | 0.2 | g, |
| Na₂HPO₄ | 0.25 | g, |
| NaCl | 0.4 | g, |
| hydroxyethyl starch 40 | 2 | g, |
| Tween 80 | 0.1 | ml, |
| injection water | to 100 | ml. |

Preparation method was same as described in Example 1.

### Example 13: (pH: 7.1)

Composition:

| | |
|---|---|
| recombinant interferon alpha-2b | 3 × 10⁸IU, |

| | | |
|---|---|---|
| NaH₂PO₄ | 0.036 | g, |
| Na₂HPO₄ | 0.274 | g, |
| NaCl | 0.4 | g, |
| dextran | 2 | g |
| Tween 80 | 0.1 | ml, |
| injection water | to 100 | ml. |

Preparation method was same as described in Example 1.

### Example 14: (pH: 7.0)

Composition:

| | |
|---|---|
| recombinant interferon alpha-2b | 3 × 10⁸IU, |

| | | |
|---|---|---|
| citric acid | 0.02 | g, |
| Na₂HPO₄ | O.25 | g, |
| NaCl | 0.4 | g, |
| hydroxyethyl starch 40 | 2 | g, |
| Tween 20 | 0.03 | ml, |
| mannitol | 1 | g |
| injection water | to 100 | ml. |

Preparation method was same as described in Example 1.

### Example 15: (pH: 7.0)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.2 | g, |
| hydroxyethyl starch 40 | 2 | g, |
| Tween 20 | 0.2 | ml, |
| mannitol | 1 | g |
| injection water | to 100 | ml. |

Preparation method was same as described in Example 1.

### Example 16: (pH: 7.0)

Composition:

| | |
|---|---|
| recombinant interferon alpha-2b | 3 × 10⁸IU, |

| | | |
|---|---|---|
| citric acid | 0.02 | g, |
| Na₂HPO₄ | 0.25 | g, |
| NaCl | 0.9 | g, |
| hydroxyethyl starch 40 | 2 | g, |
| Tween 20 | 0.05 | ml, |
| mannitol | 1 | g |
| injection water | to 100 | ml. |

Preparation method was same as described in Example 1.

Stability Test

1. The stability of the aqueous formulation according to the present invention (the formulation of the Example 12) after shaking, lighting, repeated freezing-thawing

(1) Effect of shaking on stability

The test samples were put on a shake bed (33° C., 250 rpm), sampled randomly and assayed. The results are listed as following:

a. Appearance: the sample solutions remained colorless and transparent after 7 days of shaking, protein aggregation not seen.

b. Sterility: sterile, after 7 days of shaking.

c. Biological activity (10⁸ IU): no significant change. As shown in Table 1.

TABLE 1

| | | | |
|---|---|---|---|
| Shaking time (days) | 0 | 1 | 7 |
| Biological activity | 3.41 | 3.41 | 3.41 |

d. pH: within 7.14-7.21, after 7 days of shaking.

(2) Effect of lighting on stability

The test samples were lighted (25° C., 4000 lux), sampled randomly and assayed. The results are listed in the following:

a. Appearance: the sample solutions remained colorless and transparent after 10 days of lightening, protein aggregation not seen.

b. Sterility: sterile, after 10 days of lightening.

c. Biological activity (10⁸ IU): no significant change. As shown in Table 2.

TABLE 2

| | | |
|---|---|---|
| Lighting time (days) | 0 | 10 |
| Biological activity | 3.41 | 3.32 |

d. pH: all within 7.14-7.21, after 10 days of lightening.

(3) Effect of repeated freezing-thawing on stability

The test samples were frozen in a freezer (−15° C.), and then thawed under room temperature. The above procedure was repeated for several times and the test examples were sampled and assayed randomly during the procedure. The results are listed as following:

a. Appearance: the sample solutions remained colorless and transparent after 12 times of freezing-thawing repetitions, protein aggregation not seen.

b. Sterility: sterile, after 12 times of freezing-thawing repetitions.

c. Biological activity (10⁸ IU): no significant change. As shown in Table 3.

TABLE 3

| | | | | | |
|---|---|---|---|---|---|
| Times | 0 | 3 | 6 | 9 | 12 |
| Biological activity | 3.41 | 3.32 | 3.46 | 3.41 | 3.56 |

d. pH: all within 7.14-7.21, after 12 times of freezing-thawing repetitions.

2. Storage stability of the aqueous solutions of present invention under different temperatures

(1) Stability of Examples 1-16 at 8° C.

a. Appearance: the sample solutions remained colorless and transparent after 36 months of storage at 8° C., protein aggregation not seen.

b. Sterility: sterile, after 36 months of storage at 8° C.

c. Biological activity (10⁸ IU): As shown in Table 4 (wherein the symbol “-” means “not assayed”, and “ND” means “not detectable”)

TABLE 4

| Time | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 | Ex. 13 | Ex. 14 | Ex. 15 | Ex. 16 |
|---|---|---|---|---|---|---|---|---|---|---|---|---|---|---|---|---|
| 0 | 4.1 | 1.2 | 0.60 | 3.4 | 8.5 | 5.3 | 3.9 | 3.6 | 4.2 | 3.2 | 4.1 | 3.4 | 3.2 | 3.5 | 3.4 | 3.5 |
| 1 week | — | — | — | — | — | — | 4.2 | 3.6 | 4.0 | 3.2 | 3.9 | 3.5 | 1.4 | 3.4 | 3.4 | 3.7 |
| 2 weeks | — | — | — | — | — | — | 4.0 | 3.0 | 3.9 | 3.0 | 3.8 | 3.5 | ND | 3.6 | 3.5 | 3.5 |
| 3 weeks | — | — | — | — | — | — | 3.7 | 3.2 | 4.1 | 3.0 | 4.0 | 3.5 | | 3.4 | 3.4 | 3.5 |
| 1 month | 3.9 | 1.1 | 0.57 | 3.4 | 8.3 | 5.1 | 3.8 | 2.6 | 3.0 | 2.4 | 3.9 | 3.7 | | 3.5 | 3.4 | 3.7 |
| 2 months | 3.6 | 1.1 | 0.55 | 3.4 | 8.0 | 5.2 | 3.1 | 2.3 | 2.6 | 2.5 | 3.2 | 3.4 | | 3.6 | 3.4 | 3.5 |
| 8 months | 3.3 | 1.0 | 0.52 | 3.2 | 7.8 | 5.0 | — | — | — | — | 3.2 | 3.4 | | 3.4 | 3.3 | 3.4 |
| 10 months | 3.1 | 1.1 | 0.52 | 3.3 | 7.9 | 5.1 | — | — | — | — | — | — | | 3.4 | 3.1 | 3.3 |
| 14 months | 3.0 | 1.0 | 0.50 | 3.2 | 8.0 | 5.1 | — | — | — | — | 3.0 | 3.6 | | 3.5 | 3.3 | 3.5 |
| 18 months | 3.0 | 1.0 | 0.50 | 3.3 | 7.6 | 5.0 | — | — | — | — | — | — | | 3.3 | 3.4 | 3.4 |
| 24 months | 3.0 | 1.0 | 0.49 | 3.2 | 7.6 | 4.9 | — | — | — | — | 3.0 | 3.3 | | 3.4 | 3.2 | 3.3 |
| 30 months | 3.0 | 1.0 | 0.50 | 3.1 | — | — | — | — | — | — | — | — | | 3.4 | 3.1 | 3.4 |
| 36 months | 3.0 | 0.9 | — | 3.0 | — | — | — | — | — | — | — | — | | 3.3 | 3.1 | 3.4 |

(2) Stability of Examples 1-12 at 25° C.

a. Appearance; the sample solutions remained colorless and transparent after 8 weeks of storage at 25° C., protein aggregation not seen.

b. Sterility: sterile, after 8 weeks of storage at 25° C.

c. Biological activity (10⁸ IU): as shown in Table 5 (wherein the symbol “-” means “not assayed”)

TABLE 5

| Time | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 |
|---|---|---|---|---|---|---|---|---|---|---|---|---|
| 0 week | 4.1 | 1.2 | 0.60 | 3.6 | 8.6 | 5.6 | 3.9 | 3.6 | 4.2 | 3.2 | 4.1 | 3.4 |
| 1 week | 4.0 | 1.1 | 0.55 | 3.2 | 8.3 | 5.3 | 3.8 | 3.7 | 4.0 | 4.0 | 4.0 | 3.3 |
| 2 weeks | 3.4 | 0.8 | 0.50 | 3.0 | 8.0 | 5.2 | 3.9 | 2.8 | 3.7 | 3.7 | 3.4 | 3.4 |
| 3 weeks | 3.5 | 0.9 | 0.45 | 2.9 | 8.1 | 5.0 | 3.4 | 2.6 | 3.8 | 3.8 | 3.5 | 3.6 |
| 4 weeks | 3.3 | 0.9 | 0.45 | 2.7 | 7.9 | 4.9 | 3.3 | 1.9 | 2.7 | 2.7 | 3.3 | 3.4 |
| 5 weeks | 3.2 | 1.0 | 0.40 | 2.6 | 7.9 | 4.8 | 3.2 | — | — | — | 3.1 | 3.1 |
| 8 weeks | 2.9 | 0.8 | — | — | 7.8 | — | 2.9 | — | — | — | 2.8 | 2.8 |

(3) Stability of Examples 1-12 at 37° C.

a. Appearance: the sample solutions remained colorless and transparent after 8 weeks of storage at 37° C., protein aggregation not seen.

b. Sterility: sterile, after 8 weeks of storage at 37° C.

c. Biological activity (10⁸ IU): as shown in Table 6 (wherein the symbol “-” means “not assayed”)

TABLE 6

| Time | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 |
|---|---|---|---|---|---|---|---|---|---|---|---|---|
| 0 week | 4.1 | 1.2 | 0.60 | 3.5 | 8.6 | 5.6 | 3.9 | 3.6 | 4.2 | 3.2 | 4.1 | 3.4 |
| 1 week | 3.9 | 1.2 | 0.50 | 3.0 | 8.0 | 4.6 | 3.5 | 2.4 | 3.6 | 3.0 | 3.9 | — |
| 2 weeks | 3.3 | 1.0 | 0.40 | 2.8 | 7.6 | 4.2 | 3.5 | — | — | — | 3.3 | 3.3 |
| 3 weeks | 2.7 | 0.9 | 0.25 | 2.3 | 7.0 | 3.7 | 3.0 | — | — | — | 2.7 | 3.1 |
| 4 weeks | 1.8 | 0.7 | 0.20 | 2.0 | 5.5 | — | 2.8 | — | — | — | 1.8 | 3.1 |
| 6 weeks | 1.1 | 0.5 | — | — | — | — | 2.6 | — | — | — | 1.5 | 3.0 |
| 8 weeks | 1.2 | — | — | — | — | — | 2.2 | — | — | — | 1.2 | 3.0 |

The present invention has been described in detail by way of illustration and exemplification, which is intended to facilitate the understanding of those skilled in the art. It should be clearly understood for those skilled in the art that the specific embodiments can modified and improved within the spirit and scope of the present invention. The present invention covers all of the modifications and improvements and the analogous thereof, such as applying the various different specific stabilizing systems of the invention into other types of stable interferon preparations, as well as into stabilizing other bioactive proteins.

1. A stable interferon aqueous solution formulation that may be preservative or human blood extracted ingredient free, which comprises the following components:
a. interferon alpha;
b. a buffer system to maintain pH 4.5-9.0;
c. a stabilizing agent;
d. a nonionic surfactant;
e. an osmotic pressure regulator;
f. injection water:

2. The stable interferon aqueous solution formulation of claim 1, wherein said interferon alpha comprises but not limited to interferon alpha-2.

3. The stable interferon aqueous solution formulation of claim 1, wherein said buffer system consists of citric acid and disodium hydrogen phosphate.

4. The stable interferon aqueous solution formulation of claim 1, wherein said stabilizing agent is hydroxyethyl starch 40.

5. The stable interferon aqueous solution formulation of claim 1, wherein said stabilizing agent is dextran.

6. The stable interferon aqueous solution formulation of claim 1, wherein said nonionic surfactant is polyoxyethylene sorbitan monooleate.

7. The stable interferon aqueous solution formulation of claim 1, wherein said osmotic pressure regulator is sodium chloride.

8. The stable interferon aqueous solution formulation of claim 1, wherein said osmotic pressure regulator is mannitol.

9. A stable interferon aqueous solution formulation that may be preservative or human blood extracted ingredient free, which comprises the following components:
a. 100,000-100,000,000 IU/ml interferon alpha-2b;
b. a buffer system of citric acid and disodium hydrogen phosphate to maintain pH of 4.5-9.0;
c. 5-60 mg/ml of hydroxyethyl starch;
d. 0.02-0.2 ml/100 ml of polyoxyethylene sorbitan monooleate;
e. 1-10 mg/ml of sodium chloride and 10 mgl/ml of mannitol.

10. A method for preparing the stable interferon aqueous solution formulation of claim 1, said method comprises the step of admixing interferon alpha, the buffer system to maintain pH 4.5-9.0, the stabilizing agent, the nonionic surfactant, the osmotic pressure regulator, and appropriate amount of injection water to form an aqueous formulation.

11. The method of claim 10, wherein said interferon alpha is interferon alpha-2.

12. The method of claim 10, wherein said buffer system consists of citric acid and disodium hydrogen phosphate.

13. The method of claim 10, wherein said stabilizing agent is hydroxyethyl starch 40.

14. The method of claim 10, wherein said stabilizing agent is dextran.

15. The method of claim 10, wherein said nonionic surfactant is polyoxyethylene sorbitan monooleate.

16. The method of claim 10, wherein said osmotic pressure regulator is sodium chloride.

17. The method of claim 10, wherein said osmotic pressure regulator is mannitol.

18. A use of the stable interferon aqueous solution formulation in any item of claims 1-9 in treating viral, tumorous and immune diseases.

19. A use of the combination of interferon alpha, buffer system to maintain pH 4.5-9.0, stabilizing agent, nonionic surfactants, isotonicity agent in preparing the stable interferon aqueous solution formulation that may be preservative and human serum albumin free.

20. The use of claim 19, wherein said interferon alpha is interferon alpha-2.

21. The use of claim 19, wherein said buffer system consists of citric acid and disodium hydrogen phosphate.

22. The use of claim 19, wherein said stabilizing agent is hydroxyethyl starch 40.

23. The use of claim 19, wherein said stabilizing agent is dextran.

24. The use of claim 19, wherein said nonionic surfactant is polyoxyethylene sorbitan monooleate.

25. The use of claim 19, wherein said osmotic pressure regulator is sodium chloride.

26. The use of claim 19, wherein said osmotic pressure regulator is mannitol.

27. A therapeutic method of viral, tumorous and immune diseases, said method comprises the step of administrating the stable interferon aqueous solution formulations as claimed in any one of claims 1-9 to the patients in need of such treatment.

28. A medicament comprising pre-filled syringe, wherein said pre-filled syringe is filled with an effective amount of interferon aqueous formulations as claimed in any one of claims 1-9.
